(19) United States
(12) Patent Application Publication
(10) Pub. No.: US 2015/0126466 A1
(43) Pub. Date: May 7, 2015

(54) ANTIBACTERIAL PHARMACEUTICAL COMPOSITION

(71) Applicant: OBSHCHESTVO S OGRANICHENNOY OTVETSTVENNOSTYU "VIK - ZDOROVYE ZHIVOTNYKH", Moskva (RU)

(72) Inventors: Boris Viktorovich Violin, Moscow (RU); Sergey Vyacheslavovich Semenov, Saratov (RU); Dmitriy Vladimirovich Pristenskiy, Ust-Kurdyum (RU); Aleksandr Andreevich Aniskov, Saratov (RU)

(21) Appl. No.: 14/395,483

(22) PCT Filed: Jun. 6, 2013
(86) PCT No.: PCT/RU2013/000467

§ 371 Date: Oct. 18, 2014

(30) Foreign Application Priority Data
Jun. 8, 2012 (RU) 2012123616

(51) Int. Cl. A61K 31/7052; A61K 45/06

(52) U.S. Cl. CPC A61K 31/7052; A61K 45/06
USPC 514/29

(57) ABSTRACT

The invention relates to the field of veterinary science and medicine and can be used for the prophylaxis and treatment of bacterial infections. The aim of the present invention is to design a preparation based on azithromycin in the form of a solution, having a wide spectrum of antimicrobial activity, and in the form of an injection solution ready for use not only for introduction intravenously, but also for intramuscular, subcutaneous, intrauterine and intracisternal introduction. The antibacterial pharmaceutical composition comprises azithromycin, solvents and/or cosolvents, a preservative, and an antioxidant. The antioxidant is in the form of ascorbic acid or sodium ascorbate or calcium ascorbate or palmityl ascorbate or 4-methyl-2,6-di-tert-butylphenol or tert-butylhydroquinone, 2,4,5-trihydroxy-butyrophenone or sodium metabisulfite or alpha-tocopherol or thioglycerin or combinations thereof, whereas the preservative is in the form of benzyl alcohol, parabens, chlorethone, or combinations thereof with the following ratios of components by percentage mass: 5-50% azithromycin, 0.1-0.2% antioxidant, 1-2% preservative, the remainder being solvents.

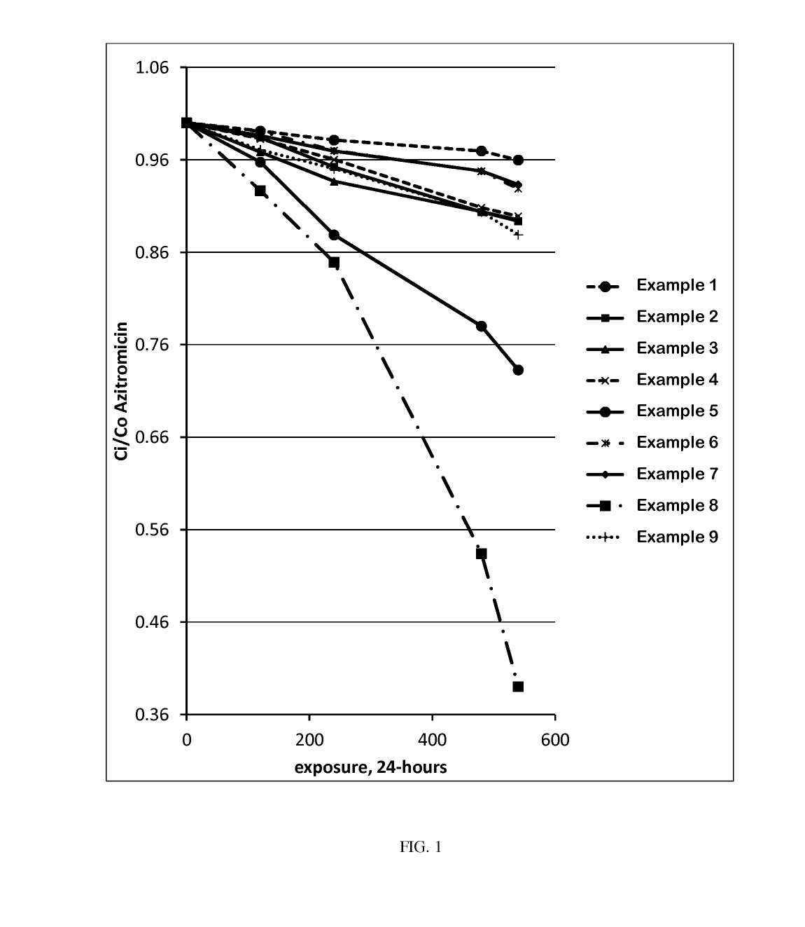

The invention relates to medicine and veterinary and can be used for the prevention and treatment of bacterial infections.

Azithromycin, (2R,3S,4R,5R,8R,10R,11R,12S,13S,14R)-13-[(2,6-dioxy-3-C-methyl-3-O-methyl-a-L-ribohexopyranozyl)oxy]-2-ethyl-3,4,10-trihydroxy-3,5,6,8,10,12,14-heptamethyl-11-[[3,4,6-trideoxy-3-(dimethylamino)-b-D-xylo-hexapyranozyl]oxy]-1-ox-6-azacyclopentadecane-15-one), is a semisynthetic antibiotic, a derivative of erythromycin. Azithromycin is active against some gram-positive bacteria: Streptococcus spp. (Groups C, F, and G, except erythromycin-resistant ones), Streptococcus pneumoniae, Streptococcus pyogenes, Streptococcus agalactiae, Streptococcus viridans, Staphylococcus epidermidis, Staphylococcus aureus; against gram-negative bacteria: Haemophilus influenzae, Moraxella catarrhalis, Bordetella pertussis, Bordetella parapertussis, Legionella pneumophila, Haemophilus ducreyi, Campylobacter jejuni, Neisseria gonorrhoeae and Gardnerella vaginalis; some anaerobes: Bacteroides bivius, Clostridium perfringens, Peptostreptococcus spp; and Chlamydia trachomatis, Chlamydia pneumoniae, Mycoplasma pneumoniae, Mycobacterium avium complex, Ureaplasma urealyticum, Treponema pallidum, Borrelia burgdorferi.

A liquid pharmaceutical formulation with azithromycin is known for ophthalmic use with the active substance content up to 10% (U.S. Pat. No. 6,277,829 B1 of Aug. 21, 2001; Antonio Asero, Grazia Mazzone, process for preparation of aqueous formulation for ophthalmic use). Another liquid azithromycin composition for topical application is also known (U.S. Pat. No. 7,064,104 B1 of Jun. 20, 2006 Jacques Luyckx, Frederic Pilotaz, Pharmaceutical composition based on macrolides for topical application in ophthalmology).

A fast-dissolving form of azithromycin for oral use is known (U.S. Pat. No. 7,572,773 B2 of Aug. 11, 2009 Aleksandar Danilovsky, Nezevie Zdravka, single dose fast dissolving azithromycin).

Azithromycin salts with malonic acid have been isolated. These compounds can be used for making fast-dissolving forms of azithromycin for per oral applications (U.S. Patent No 2009/031375 A1 B2 of Dec. 24, 2009 Bo Sung Kwon, Eun Sook Kim, Hee Cheol Kim, Sangmin Yun, Myoung-sill Ko, Tae Hun Song, Han Kyong Kim, Kwee Hyun Suh, Gwansum Lee, crystalline azithromycin L-malate monohydrate and pharmaceutical composition containing same).

Various solid pharmaceutical compositions of azithromycin for oral use are known (U.S. Patent No 2007/0185194 A1 of Aug. 9, 2007 Kamal Mehta, Rajeev Shankar Mathur, Sujata Paul, Sanjeev Kumar Sethi, Rajiv Malik, stable oral compositions of azithromycin monohydrate; U.S. Patent No 2007/01851 A1 of Aug. 9, 2007 Kamal Mehta, Rajeev Shankar Mathur, Sujata Paul, Sanjeev Kumar Sethi, Rajiv Malik, stable oral compositions of azithromycin monohydrate; U.S. Patent No 2005/0106239 A1 of May 19, 2005 Ruth Tenengauzer, Joseph Schwarz, Julia Hrakovsky, Tania Lessen, Lev Khondo, Mathi Mathivanan, Claude Singer, Michal Pesachovich, stabilized azithromycin compositions; U.S. Patent No 2008/0096831 A1 of Apr. 24, 2008 Mohsen Sadatrezaei, Pablo Davila, Gary Barbera, stabilized azithromycin composition).

All the above compositions are intended for oral or topical use.

A solid dosage form comprising lyophilized azithromycin is known (U.S. Pat. No. 0,116,336 of Jul. 1, 2006 Byung But Woo, K. Keith Knwok, Kang Yong Yang, lyophilized azithromycin formulation). There exists a lyophilized form of azithromycin under the commercial name “Sumamed”, Pliva production.

This dosage form is a sterile powder to prepare injection solution immediately prior to use because the antibiotic in solution rapidly decomposes.

The liquid injectable form of azithromycin, comprising ethyl alcohol, cosolvents (polyethanediol, propylene glycol), a pH regulator is the most similar to our proposed solution (see patent CN 1613453 “Azithromycin injection and its preparation”).

However, this formulation contains ethanol; therefore it cannot be administered intramuscularly or subcutaneously because this would lead to tissue necrosis. It is only used intravenously and in dilute aqueous solution. Hence, it is not a ready-to-use injectable solution but a concentrate to be added to a solution for intravenous infusion.

The present invention is aimed at the development of an azithromycin preparation as a solution with a broad spectrum of antimicrobial activity, as a ready-to-use injection solution for intramuscular, subcutaneous, intrauterine and intracisternal administration as well as for intravenous administration.

Our technical result is a high physicochemical stability (at least 18 months) of our liquid dosage form, while providing security for the treatment and prevention of diseases caused by pathogenic microorganisms.

The problem is solved by that our antibacterial pharmaceutical composition comprising azithromycin, solvents and/or cosolvents, a preservative, according to our invention, additionally contains an antioxidant (ascorbic acid or sodium ascorbate or calcium ascorbate or palmityl ascorbate or 4-methyl-2,6-di-tert-butylphenol or tert-butylhydroquinone or 2,4,5-trihydroxybutyrophenone or sodium metabisulphite or alpha-tocopherol or thioglycerol or combinations thereof), while benzyl alcohol, parabens, chlorethone or combinations thereof act as preservatives, with the following component ratio, wt. %: azithromycin 5-50%; antioxidant 0.1-0.2%, preservative 1-2%; the balance, solvents. Organic solvents (dimethylacetamide or dimethyl sulfoxide or N-methyl-2-pyrrolidone or N-ethyl-2-pyrrolidone or N-ethoxy-2-pyrrolidone or N-octyl-2-pyrrolidone or 3,3-dimethyl-2-pyrrolidone, propylene glycol or transcutol (monoethyl ether of diethylene glycol) or benzyl alcohol, or combinations thereof) or water or combinations of organic solvents with water can be used as solvents or cosolvents. The composition may additionally contain a pH regulator in an amount of up to 5%; a solubilizer (polyethylene glycol 660, 12-hydroxy stearate or poloxamer F 68 or poloxamer 127 or polyoxyethylene-glycerol-triricinoleate or polyoxyethylene-glycerol-trihydroxy stearate or polyoxyethylene sorbitan monooleate, or a combination thereof) in an amount within 1-30%, and an anesthetic (up to 2.0%): novocaine or sevicaine or bupivacaine or ropivacaine or lidocaine, or mixtures thereof can be used as anesthetics.

In the composition, the active substance is dissolved in a mixture of organic solvent and water with substances to stabilize the acidity of the solution added. The ratio of the organic solvent, water, and pH (acidity) regulators is selected in such a way that the physicochemical stability of the solution was as high as possible with the minimum degradation degree of the dissolved azithromycin. The organic solvent and pH adjuster are selected for the lowest toxicity when intramuscular or subcutaneous administration.

The invention is illustrated by a graph which shows data on the degradation of azithromycin solutions produced by several recipes listed below (Examples 1-9).

The developed formulation is stable solution of the active ingredient (azithromycin). Water, dimethyl sulfoxide or 2-pyrrolidinone or N-methyl-2-pyrrolidone or N-ethyl-2-pyrrolidone and/or N-ethoxy-2-pyrrolidone or N-octyl-2-pyrrolidone or dimethylacetamide or 3,3-dimethyl-2-pyrrolidone or propylene glycol or transcutol (monoethyl ether of diethylene glycol), or combinations thereof are used as solvent and/or cosolvent. Citric acid or malic acid or benzoic acid or succinic acid, or combinations thereof are used as pH adjusters. Benzyl alcohol, parabens, monochlorethone, or combinations thereof are used as preservatives. Ascorbic acid or sodium ascorbate or calcium ascorbate or palmityl ascorbate or 4-methyl-2,6-di-tert-butylphenol or tert-butyl hydroquinone or 2,4,5-trihydroxybutyrophenone or sodium metabisulphite or thioglycerol or tocopherol acetate, or combinations thereof are used as antioxidants.

The dosage form may include a solubilizer. Cremophor EL or Cremophor ELP (polyoxyethylene-glycerol-triricinoleate) or Cremophor RH40 (polyoxyethylene-glycerol-trihydroxystearate) or Tween 80 (polyoxyethylene sorbitan monooleate) or Solutol HS 15 (polyethylene glycol-660-12-hydroxystearate) or Poloxamer F68 or Poloxamer F127 (polyoxyethylene-polyoxypropylene-block-copolymer), or combinations thereof are used as solubilizers. The dosage form may also include an anesthetic-lidocaine hydrochloride (up to 0.5%).

Methods for assessing the physicochemical stability of azithromycin solutions The content of azithromycin in solution was measured by HPLC using the method prescribed by RF State Pharmacopoeia XII, FS “Azithromycin”.

To justify our selection of the most optimal solvent, the solubility of azithromycin in various organic solvents was measured (Table 1).

TABLE 1
Azithromycin solubility in organic solvents.

| Solvent | Maximum azithromycin conc., % |
|---|---|
| Dimethylsulfoxide | 26.4 |
| 2-Pyrrolidone | 28.9 |
| N-methyl-2-pyrrolidone | 31.2 |
| N-ethyl-2-pyrrolidone | 25.8 |
| N-ethoxy-2-pyrrolidone | 23.3 |
| N-octyl-2-pyrrolidone | 21.5 |
| Dimethylacetamide | 32.1 |
| 3,3-dimethyl-2-pyrrolidone | 26.4 |
| Propylene glycol | 13.4 |
| Transcutol | 10.2 |

N-methyl-2-pyrrolidinone and dimethylacetamide are most optimal solvents. When dissolved in these solvents in the presence of the abovementioned pH regulators, the solubility of azithromycin increases up to 50% (see Example 9).

Method of Preparation

Azithromycin is suspended in an appropriate solvent or a solvent mixture, and then a pH adjustor, a solubilizer, an anesthetic, a preservative, and an antioxidant are added.

Below are several compositions of our dosage forms to illustrate the optimal ratio of the components:

### EXAMPLE 1

A composition with an anesthetic is provided.

| Component | Amount, wt. % |
|---|---|
| Azithromycin | 10.0 |
| Lidocaine hydrochloride | 0.1 |
| N-methyl-2-pyrrolidone | 50.0 |
| Citric acid | 3.2 |
| Benzyl alcohol | 1.0 |
| Methyl-2,6-di-tert-butylphenol | 0.1 |
| Water | Up to 100 |

### EXAMPLE 2

A formulation is provided with a solubilizer (polyoxyethylene-glycerol-triricinoleate) and the corresponding amounts of other components.

| Component | Amount, wt. % |
|---|---|
| Azithromycin | 10.0 |
| Dimethylacetamide | 30.0 |
| Citric acid | 3.2 |
| Polyoxyethylene-glycerol-triricinoleate | 15.0 |
| Benzyl alcohol | 1.0 |
| Sodium metabisulfite | 0.1 |
| Water | Up to 100 |

### EXAMPLE 3

A formulation is provided with the maximum amount of the solubilizer and appropriate amounts of other components.

| Component | Amount, wt. % |
|---|---|
| Azithromycin | 10.0 |
| Dimethylacetamide | 35.0 |
| Citric acid | 3.0 |
| Polyoxyethylene-glycerol-triricinoleate | 30.0 |
| Benzyl alcohol | 1.0 |
| Methyl-2,6-di-tert-butylphenol | 0.1 |
| Water | Up to 100 |

Increasing the amount of the solubilizing agent above 30% leads to instability of the solution (delamination and precipitation).

### EXAMPLE 4

A formulation with the maximum amount of the organic solvent and appropriate amounts of other components is provided.

| Component | Amount, wt. % |
|---|---|
| Azithromycin | 20.0 |
| Dimethylacetamide | 74.0 |
| Citric acid | 1.6 |
| Benzyl alcohol | 1.0 |
| Methyl-2,6-ditert butylphenol | 0.1 |
| Water | Up to 100 |

An increased amount of the organic solvent (above 80%) leads to enhanced toxic properties of the dosage form.

### EXAMPLE 5

A formulation with the maximum amount of organic solvent and appropriate amounts of other components, without pH control, is provided.

| Component | Amount, wt. % |
|---|---|
| Azithromycin | 20.0 |
| Dimethylacetamide | 74.0 |
| Benzyl alcohol | 1.0 |
| Methyl-2,6-di-tert-butylphenol | 0.1 |
| Water | Up to 100 |

The elimination of the regulator from the formulation affects the stability of the solution (rapid darkening, a drop of the active ingredient concentration); in addition, an increased amount of the organic solvent (above 70%) leads to increased toxic properties of the dosage form.

### EXAMPLE 6

A formulation is provided with poloxamer F127 as a solubilizer, with no addition of an organic cosolvent, and with appropriate amounts of other components.

| Component | Amount, wt. % |
|---|---|
| Azithromycin | 5.0 |
| Citric acid | 3.2 |
| Poloxamer F127 | 5.0 |
| Benzyl alcohol | 1.0 |
| Sodium meta bisulfite | 0.1 |
| Water | Up to 100 |

A reduced amount of the solubilizer (below 5.0%) entails a decreased stability of the dosage forms (precipitation); besides, a reduced amount of the active agent leads to a decreased therapeutic efficacy of the formulation.

### EXAMPLE 7

A formulation is provided with Poloxamer F127 as a solubilizer, with no addition of an organic cosolvent, and with more azithromycin than in example 6:

| Component | Amount, wt. % |
|---|---|
| Azithromycin | 10.0 |
| Citric acid | 3.2 |
| Poloxamer F127 | 8.0 |
| Benzyl alcohol | 1.0 |
| Sodium metabisulfite | 0.1 |
| Water | Up to 100 |

An increased azithromycin content (above 10.0%), when Poloxamer F127 is used as a solubilizer, leads to an increased viscosity of the solution at temperatures below +15° C.

### EXAMPLE 8

A formulation is provided with the maximum amount of a pH regulator and appropriate amounts of other components.

| Component | Amount, wt. % |
|---|---|
| Azithromycin | 10.0 |
| Dimethylacetamide | 35.0 |
| Citric acid | 5.0 |
| Benzyl alcohol | 1.0 |
| Ascorbic acid | 0.1 |
| Water | Up to 100 |

An increased amount of a pH regulator (above 5%) results in an increased acidity of the solution, and enhanced the local irritating effect of the dosage form enhances, and the degradation rate of the main component rises.

### EXAMPLE 9

A formulation is provided with the maximum amount of the active substance and appropriate amounts of other components.

| Component | Amount, wt. % |
|---|---|
| Azithromycin | 50.0 |
| Dimethylacetamide | 43.9 |
| Citric acid | 5.0 |
| Benzyl alcohol | 1.0 |
| Ascorbic acid | 0.1 |
| Water | Up to 100 |

When the concentration of azithromycin becomes more than 50%, the solution becomes insufficiently stable, and solutions with the concentration of the active substance within 30-50% are viscous enough, which deteriorates their operating properties (performance).

### EXAMPLE 10

A formulation is provided where propylene glycol is used as an organic solvent.

| Component | Amount, wt. % |
|---|---|
| Azithromycin | 10.0 |
| Propylene glycol | 70.0 |
| Benzoic acid | 3.2 |
| Benzyl alcohol | 1.0 |
| Ascorbic acid | 0.1 |
| Water | Up to 100 |

The forms with propylene glycol as the main solvent are less stable (precipitation).

### EXAMPLE 11

A formulation is provided where transcutol is used as an organic solvent.

| Component | Amount, wt. % |
|---|---|
| Azithromycin | 10.0 |
| Transcutol | 60.0 |
| Benzoic acid | 3.2 |
| Benzyl alcohol | 1.0 |
| Ascorbic acid | 0.1 |
| Water | Up to 100 |

### EXAMPLE 12

A formulation is provided where dimethylacetamide and benzyl alcohol are used as organic solvents.

| Component | Amount, wt. % |
|---|---|
| Azithromycin | 10.0 |
| Dimethylacetamide | 30.0 |
| Benzoic acid | 3.2 |
| Benzyl alcohol | 20.0 |
| Ascorbic acid | 0.1 |
| Water | Up to 100 |

The forms with benzyl alcohol (or other alcohols) as a cosolvent are characterized by lower stability (color changes of the solution and degradation of the active ingredient).

A stability study was carried out for samples stored at ambient temperature (17-26° C.). The results are shown in Table 2.

TABLE 2

| | Exposure, days | | | | |
|---|---|---|---|---|---|
| | 0 | 120 | 240 | 480 | 540 |
| Example No | Azithromycin concentration, % | | | | |
| 1 | 10.17 | 10.08 | 9.98 | 9.86 | 9.76 |
| 2 | 10.05 | 9.89 | 9.57 | 9.08 | 8.98 |
| 3 | 10.10 | 9.78 | 9.46 | 9.13 | 9.04 |
| 4 | 20.68 | 20.32 | 19.86 | 18.78 | 18.59 |
| 5 | 20.46 | 19.59 | 17.98 | 15.96 | 14.99 |
| 6 | 5.34 | 5.29 | 5.18 | 5.06 | 4.96 |
| 7 | 10.17 | 10.03 | 9.86 | 9.64 | 9.49 |
| 8 | 10.22 | 9.47 | 8.68 | 5.46 | 3.99 |
| 9 | 50.54 | 49.06 | 48.03 | 45.67 | 44.42 |

From these data it follows that the dosage forms made according to the above formulations satisfy the requirements of physicochemical stability, Formulations 1-4 being the most optimal. Adding an anesthetic to the composition does not alter the physicochemical properties of the dosage form.

From the graph it follows that addition of inadequate amounts of a pH adjuster leads to instability of the solution (a drop of the active ingredient concentration, Example 5) as well as the use of its excessive amounts (Example 8). Ignoring an organic solvent in the composition of the injectable solution with simultaneous addition of an appropriate solubilizer (poloxamer 127 F) renders no effect on the solution stability (Examples 6, 7), and the prepared dosage form lacks drawbacks due to the presence of an organic solvent (lower toxicity). Inclusion of a solubilizer into the solution, with the simultaneous usage of an organic solvent, has no effect on the stability (Examples 1-4), but the presence of a surfactant may improve the pharmacological properties of the resulting formulations. An increased azithromycin content (up to 50%) (Example 9) causes the usage of an organic solvent with a pH adjuster only, and the use of surfactants is excluded due to the high viscosity of the forms with a solubilizer in their composition.

1.-6. (canceled)

7. An antibacterial pharmaceutical composition, comprising:
azithromycin;
solvents or co-solvents;
a preservative, wherein the preservative is selected from the group consisting of benzyl alcohol, parabens, chlorethone and combinations thereof;
an antioxidant, wherein the antioxidant is selected from the group consisting of ascorbic acid, sodium ascorbate, calcium ascorbate, palmityl ascorbate, 4-methyl-2,6-di-tert-butylphenol, tert-butylhydroquinone, 2,4,5-trihydroxybutyrophenone, sodium metabisulphite, alpha-tocopherol, thioglycerol, and combinations thereof;
wherein the azithromycin is present in a weight percentage in the range of 5-50%;
wherein the antioxidant is present in a weight percentage in the range of 0.1-0.2%;
wherein the preservative is present in a weight percentage in the range of 1-2%; and
wherein a balance of weight percentage of the composition comprises the solvents or co-solvents.

8. The composition according to claim 7, wherein the solvents or co-solvents include organic solvents or co-solvents, water, or a combination of organic solvents or co-solvents and water, wherein the organic solvents or co-solvents are selected from the group consisting of dimethylacetamide, dimethyl sulfoxide, N-methyl-2-pyrrolidone, N-ethyl-2-pyrrolidone, N-ethoxy-2-pyrrolidone, N-octyl-2-pyrrolidone, 3,3-dimethyl-2-pyrrolidone, propylene glycol, transcutol (monoethyl ether of diethylene glycol), benzyl alcohol and combinations thereof.

9. The composition according to claim 7, further comprising a pH adjuster, wherein the pH adjuster is present in a weight percentage up to 5%.

10. The composition according to claim 7, further comprising a solubilizer, wherein the solubilizer is selected from the group consisting of polyethylene glycol 660 12-hydroxy-stearate, poloxamer F 68, poloxamer F 127, polyoxyethylene-glycerol-triricinoleate, polyoxyethylene-glycerol-trihydroxystearate, polyoxyethylene sorbitan monooleate and combination thereof, wherein the solubilizer is present in a weight percentage in the range of 1-30%.

11. The composition according to claim 7, further comprising an anesthetic, wherein the anesthetic is present in a weight percentage up to 2.0%.

12. The composition according to claim 11, wherein the anesthetic is selected from the group consisting of novocaine, sevicaine, bupivacaine, ropivacaine, lidocaine and combinations thereof.
